(19) United States
(12) Patent Application Publication
(10) Pub. No.: US 2014/0052048 A1
(43) Pub. Date: Feb. 20, 2014

(54) TAMPON APPLICATOR

(71) Applicants: Kenta Taniguchi, Kagawa (JP); Yukihiro Ito, Kagawa (JP); Kouichi Yamaki, Kagawa (JP)

(72) Inventors: Kenta Taniguchi, Kagawa (JP); Yukihiro Ito, Kagawa (JP); Kouichi Yamaki, Kagawa (JP)

(73) Assignee: Unicharm Corporation, Shikokuchuo-shi Ehime (JP)

(21) Appl. No.: 13/985,826

(22) PCT Filed: Feb. 16, 2012
(86) PCT No.: PCT/JP2012/001044

§ 371 Date: Oct. 24, 2013

(30) Foreign Application Priority Data
Feb. 18, 2011 (JP) 2011-033315

(51) Int. Cl. A61F 13/26

(52) U.S. Cl. CPC A61F 13/266
USPC 604/15

(57) ABSTRACT

An tampon applicator includes an outer tube which contains an absorber therein, of which the one side is provided with a push-out aperture by which the absorber is to be pushed out, and of which the other side is provided with a grip tube unit; and an inner tube which is movable towards into the outer tube to push the absorber to the outside through the push-out aperture; and a collar unit extending radially outwards from an outer circumferential surface of the grip tube unit of the outer tube is included, and having a light transmissive means that enables the grip tube unit side to be visually confirmed from the push-out aperture side of the outer tube via the collar unit.

### TECHNICAL FIELD

The present invention relates to a tampon applicator.

### BACKGROUND ART

Conventionally, a sanitary tampon with an applicator is provided. A tampon applicator includes an outer tube and an inner tube. Inside the outer tube is contained an absorber having a withdrawal string. When using a sanitary tampon, the user inserts the outer tube inside the vagina while gripping the outer tube, and then presses the inner tube towards the outer tube. When the inner tube is pressed towards the outer tube, the absorber is pushed out from the outer tube and is disposed inside the vagina. However, when using a sanitary tampon, if the user pushes out the absorber when the outer tube has not been inserted up to an appropriate depth, the absorber is not disposed at an appropriate position inside the vagina.

Patent Document 1 describes a tampon applicator made in view of this problem. The Tampon applicator includes a collar unit in a curved shape extending out in the circumference of the outer tube. When using the sanitary tampon, the user grips the outer tube via the collar unit, and inserts the outer tube inside the vagina. When the outer tube is inserted up to an appropriate depth, the collar unit comes in contact with the vaginal opening. Therefore, the user can insert the outer tube up to the appropriate depth. When the user pushes out the absorber with the outer tube inserted up to an appropriate depth, the absorber is disposed at an appropriate position inside the vagina.

### CITATION LIST

### Patent Literature

PTL 1: Japanese Patent No. 3217617

### SUMMARY OF INVENTION

### Technical Problem

However, the applicants noted the following potential problems as regard the aforementioned tampon applicator. When using such a tampon, the user inserts the outer tube inside the vagina by facing the tip of the tampon applicator towards the vaginal opening while opening the genital labial with one hand, and holding the inner tube, grip tube unit, and collar unit with the other hand. At this time, when the tampon applicator is seen from the user, the hand holding the inner tube is positioned posterior to the collar unit. Therefore, the user may not be able to visually confirm the hand holding the inner tube of the tampon, and was thus unable to press the inner tube smoothly towards the outer tube, and unable to perform the operation of pressing the inner tube towards the outer tube. As a result, it may be difficult to dispose the absorber at an appropriate position inside the vagina.

Thus, the present invention has been made in view of the above problems, and its purpose is to provide a tampon applicator that can easily dispose an absorber at an appropriate position inside the vagina.

### Solution to Problem

The feature of the present invention includes an outer tube (outer tube 2) which contains an absorber (absorber 4) therein, of which the one side is provided with a push-out aperture (push-out aperture 8) by which the absorber is to be pushed out, and of which the other side is provided with a grip tube unit (grip tube unit 7); an inner tube (inner tube 3) which is inserted inside the grip tube unit, and which is movable towards into the outer tube to push the absorber to the outside through the push-out aperture; and a collar unit (collar unit 11) extending radially outwards from an outer circumferential surface of the grip tube unit of the outer tube is included, and having a light transmissive means that enables the grip tube unit side of the outer tube to be visually confirmed from the push-out aperture side of the outer tube via the collar unit.

### Advantageous Effects of Invention

According to the present invention, because the user can visually confirm the dorsal side through the light transmissive means, she can visually confirm the hand holding the inner tube and grip tube unit (hand operating the tampon applicator). Because the user can understand the operating status, she can push out the absorber smoothly and can dispose the absorber at an appropriate position inside the vagina.

### BRIEF DESCRIPTION OF DRAWINGS

FIG. 1 is a perspective view of a tampon applicator according to a first embodiment of the present invention.

FIG. 2 is a plan view of the tampon applicator shown in FIG. 1.

FIG. 3 is a schematic front view showing a form of usage of the tampon applicator shown in FIG. 1.

FIG. 4 is a plan view of a collar unit according to a first modification.

FIG. 5 is a plan view of a collar unit according to a second modification.

FIG. 6 is a plan view of a collar unit according to a third modification.

### DESCRIPTION OF EMBODIMENTS

### First Embodiment

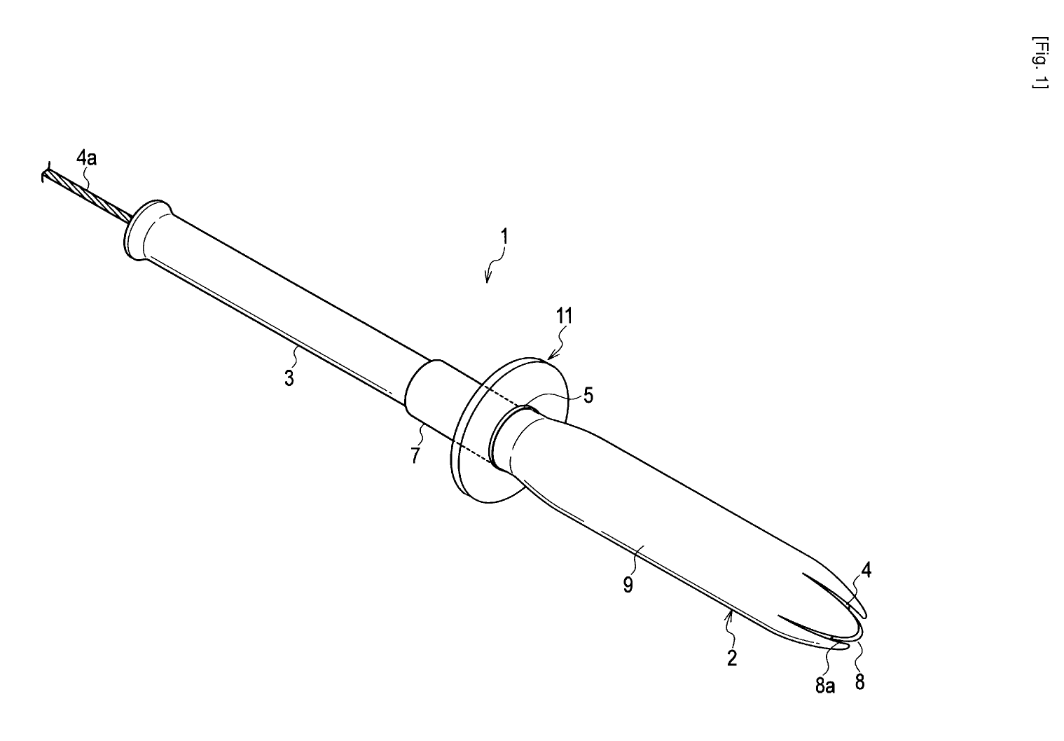

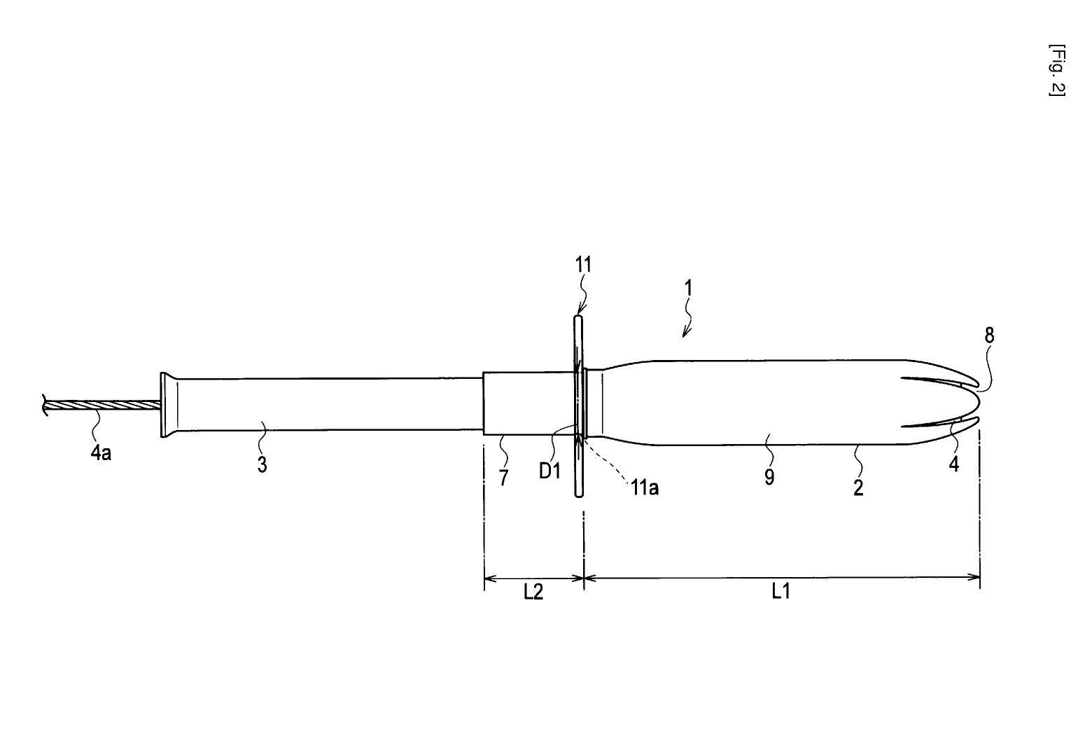

The tampon applicator according to the first embodiment of the present invention is explained with reference to FIG. 1 and FIG. 2. FIG. 1 is a perspective view showing the entire tampon applicator according to the first embodiment, and FIG. 2 is a plan view of the tampon applicator shown in FIG. 1.

A tampon applicator 1 has an outer tube 2 and an inner tube 3. The outer tube 2 and the inner tube 3 are cylindrical in shape having a hollow portion therein. The cross-sectional shape of the outer tube 2 and the inner tube 3 is a precise circle. The outer tube 2 and the inner tube 3 are entirely formed by a polyolefin resin such as polyethylene and polypropylene, or by a cardboard whose surface is laminated with a polyolefin film. In the present embodiment, the outer tube 2 and the inner tube 3 are formed by injection molding by mixing together polyethylene and polypropylene, 1% or more of a pigment, and 1% or more of a lubricant. Although the cross-sectional shape of the outer tube 2 and the inner tube 3 according to the present embodiment is a precise circle, the cross-sectional shape of the outer tube 2 and the inner tube 3 according to the present invention can also be of any shape, for example, an elliptical shape as long as it can be inserted easily inside the vagina.

Inside the outer tube 2 is contained an absorber 4 as a tampon. A withdrawal string 4a is connected to the absorber 4. The withdrawal string 4a is inserted inside the inner tube 3, extends from the end of the absorber 4, and the inserted end is extracted out from the inner tube 3. By pulling the withdrawal string 4a during use, the absorber 4 can be pulled out from inside the body.

A push-out aperture 8 from which the absorber 4 is pushed out is provided at one end of the outer tube 2. In the push-out aperture 8, a petal body 8a that is to be deformed radially outwards when the absorber 4 is pushed out is formed. The petal body 8a is always closed at an edge of the push-out aperture 8, and when the absorber 4 is pushed out by the inner tube 3, it gets flared by the absorber 4 and opens up. Thus, the absorber 4 is pushed out from the outer tube 2, and can be inserted inside the body.

At the other end of the outer tube 2, a grip tube unit 7 that is to be gripped by fingers during the movement operation of the outer tube 2 and the inner tube 3 is provided. The grip tube unit 7 has a smaller diameter than the diameter of the outer tube main body 9 between the push-out aperture 8 and the grip tube unit 7. A tip portion of the inner tube 3 is inserted in the grip tube unit 7, and the tip surface of the inserted inner tube 3 faces the absorber 4. The end part of the outer tube main body 9 at the side of the grip tube unit 7 is narrowed to almost the same size in diameter as the grip tube unit 7, and has a protrusion 5. The protrusion 5 is in contact with the collar unit 11 described later, and restrains the movement of the collar unit 11 such that the collar unit 11 does not move towards the outer tube main body 9 side.

In the grip tube unit 7, a collar unit 11 that extends radially outwards from the grip tube unit 7 is installed. The collar unit 11 is in contact with the protrusion 5. In the collar unit 11, a hole 11a in which the grip tube unit 7 is inserted is formed. The diameter of the hole 11a is desired to be larger than the diameter of the circumference of the grip tube unit 7 or the same as the diameter of the circumference of the grip tube unit 7. Furthermore, the inner diameter D1 of the hole 11a of the collar unit 11 is smaller than the outer diameter of the protrusion 5. Therefore, the collar unit 11 can be moved in the circumferential direction and the axial direction of the grip tube unit 7. Furthermore, the movement of the collar unit 11 towards the outer tube main body 9 is restrained by the protrusion 5.

In the tampon applicator 1 of the present invention, the inner diameter D1 of the collar unit 11 is 9.9 mm. The outer diameter of the outer tube main body 9 is 13.7 mm, the outer diameter of the grip tube unit 7 is 9.4 mm, and the outer diameter of the protrusion 5 is 10.4 mm. Furthermore, the length L1 from the end of the outer tube 2 at the side of the push-out aperture 8 up to the collar unit 11 (see FIG. 2) is 50 mm, and the length L2 from the surface on the tip side of the collar unit 11 up to the end of the outer tube 2 of the grip tube unit 7 side (see FIG. 2) is 10 mm. Because the length L2 from the surface of the tip side of the collar unit 11 up to the end of the outer tube 2 on the side of the grip tube unit 7 is the length of the portion that the user grips with fingers at the time of use, it is configured to be 10 mm or more for the ease of holding by the user. Furthermore, the thickness of the collar unit 11 is 1.5 mm.

The external shape of the collar unit 11 is circular in a plan view. The total light transmittance of the collar unit 11 is configured to be 63% or more. The collar unit 11 is entirely semi-transparent and the configuration is such that the dorsal side can be visually confirmed (visually recognizable) via the collar unit 11 from the tip side (i.e., the side of the push-out aperture 8). That is, the entire collar unit 11 functions as a light transmissive means. The total light transmittance can be controlled by the amount of titanic oxide contained in a resin material configuring the collar unit 11 or by forming fine convexoconcaves on the surface of the collar unit 11. Specifically, it is possible to reduce the total light transmittance by increasing the contained amount of titanic oxide or by forming fine convesoconcaves.

Due to the fact that the total light transmittance of the collar unit 11 is 63% or more, the operating hand can be visually confirmed via the collar unit 11. Furthermore, the total light transmittance of the collar unit 11 is configured to be 90% or less. If the total light transmittance of the collar unit 11 becomes too high, the transparency becomes high, and it might be difficult for the user to recognize the collar unit. However, according to a collar unit with a total light transmittance of 90% or less, the user can certainly recognize the collar unit. As used herein, “semi-transparency” means that the total light transmittance is 63% or more and 90% or less, and “transparent” means that the total light transmittance is more than 90%.

The collar unit 11 is configured from a polyolefin resin, an elastomer, a paper, or any other material. The collar unit 11 according to the present embodiment is configured from a polyethylene and polypropylene resin.

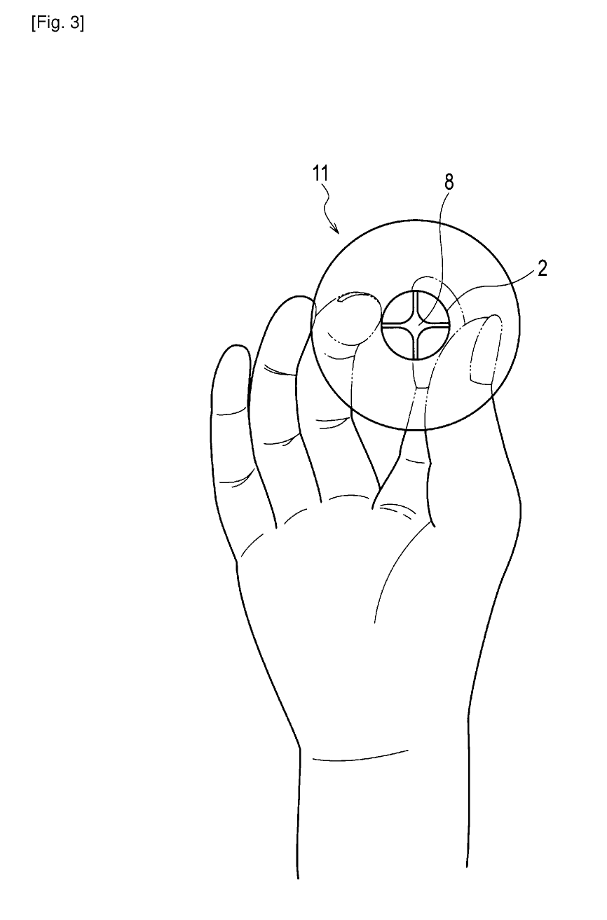

FIG. 3 is a schematic front view showing a form of usage of the tampon applicator. FIG. 3 schematically shows the state in which the tampon applicator 1 is gripped by the user is visually confirmed from the tip (push-out aperture 8) side of the outer tube 2. Due to the fact that the entire collar unit 11 is semi-transparent, the user can visually confirm the inner tube 3 and the hand holding the grip tube unit 7 (hand operating the tampon applicator) via the collar unit 11. Thus, when the inner tube 3 is pressed towards the outer tube 2, the operating status can be observed, and the absorber can be disposed at an appropriate position inside the vagina by pushing out the absorber 4 smoothly.

The diameter of the collar unit 11 is configured to be of a length extending towards the outside by 2 mm from at least the inner diameter of the hole from the viewpoint of securing the strength around the hole 11a. Finally, the diameter of the collar unit 11 is configured to be at least 15 mm or more when the collar unit 11 and the vaginal opening come in contact, from the viewpoint of preventing the bodily fluid from adhering to the finger.

Furthermore, because the collar unit 11 can rotate with respect to the grip tube unit 7, the outer tube 2 can be inserted inside the body while maintaining an appropriate angle by rotating the collar unit 11 with respect to the outer tube 2 when the outer tube 2 is inserted inside the body while gripping the collar unit 11. Specifically, the angle and position of the hand holding the tampon applicator 1 change in the state when the push-out aperture 8, which is the tip of the outer tube 2, is in contact with the vaginal opening, and in the state when the outer tube 2 is inserted inside the vagina and the collar unit 11 is in contact with the vaginal opening. At this point, by rotating the collar unit 11 with respect to the outer tube 2, the relative angle between the collar unit 11 and the outer tube 2 can be changed in accordance with the change in the position and angle of the hand with respect to the tampon applicator. Thus, even when the angle and position of the hand holding the tampon applicator 1 change, the user can smoothly insert the Tampon applicator by exerting an appropriate force thereto.

Furthermore, the collar unit 11 in the present embodiment is formed by performing injection molding of a thermoplastic resin using a mold. When performing injection molding, the collar unit 11 may be molded together with the outer tube 2, or may be molded separately and then fitted through the single-unit forming step during the manufacturing process. Furthermore, the collar unit 11 may be configured to be attached and detached freely with respect to the outer tube 2, and to be fitted by the user before use.

Subsequently, a form of usage of a tampon applicator thus configured is explained. When using the absorber 4, the user inserts the outer tube 2 up to an appropriate position inside the vagina. When the outer tube 2 is inserted up to the appropriate position, the flat surface of the back side (outer tube main body side) of the collar unit 11 is disposed in the proximity of the vaginal opening. If a further attempt is made to insert the outer tube 2, the collar unit 11 comes in contact with the body of the user. That is, the collar unit 11 exhibits the function of a stopper, and the user can understand that she has managed to insert the outer tube 2 up to the appropriate position. Furthermore, because the collar unit 11 is disposed between the vaginal opening and the fingers when the outer tube 2 has been inserted up to the appropriate position, the contamination of the fingers of the user by the bodily fluid, such as menstrual blood, can be prevented.

Next, due to pressing of the inner tube 3 towards the outer tube 2 side by the user after inserting the outer tube 2 up to a predetermined position inside the vagina, the absorber 4 is pushed out from the push-out aperture 8 of the outer tube 2, and the absorber 4 is disposed at an appropriate position inside the vagina. Thus, by including the collar unit 11, the absorber 4 can be disposed easily at the appropriate position without the fingers of the user touching the proximity of the vaginal opening. By disposing the absorber 4 at the appropriate position, the feeling of discomfort at during use can be minimized, and the user can perceive the use of the tampon as being comfortable.

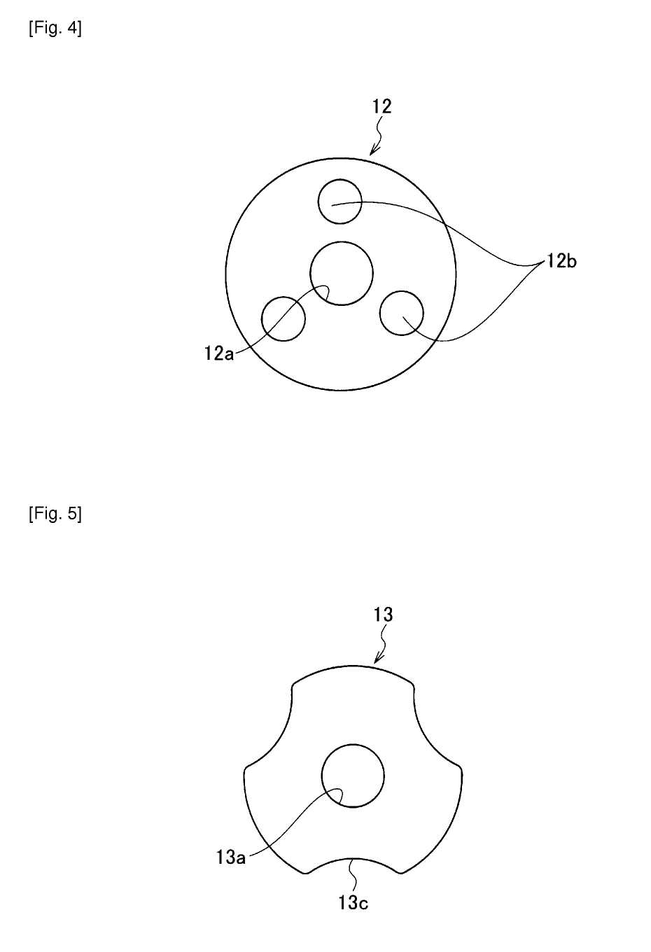

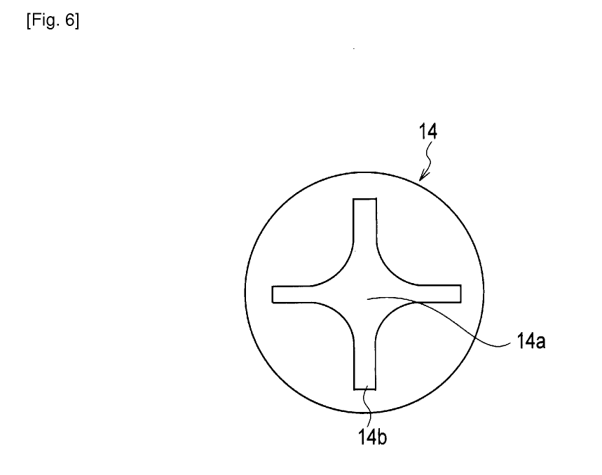

The collar unit 11 according to the first embodiment is configured entirely as a light transmissive means. However, the collar unit 11 according to the present embodiment is not limited to this configuration and only a part of the collar unit can be configured as a light transmissive means. Subsequently, a collar unit according to modifications is explained. FIG. 4 is a plan view of a collar unit 12 according to a first modification, FIG. 5 is a plan view of a collar unit 13 according to a second modification, and FIG. 6 is a plan view of a collar unit 14 according to a third modification. In the following explanation of the modifications, the description about the same configuration as the first embodiment is omitted.

The external shape of the collar unit 12 according to the first modification is almost circular in a plan view. In the center of the collar unit 12, a hole 12a in which the grip tube unit 7 is inserted is formed. At least one aperture is provided in the collar unit 12 besides the hole 12a. For example, around the hole 12a, apertures 12b are formed as the light transmissive means. By providing the apertures 12b, the user can visually confirm the hand operating the inner tube via the collar unit 11 even when the total light transmittance of the collar unit 11 is 63% or less.

The size of the apertures 12b may be configured so as to enable visual confirmation of the operating hand via the aperture 12b, and specifically is preferably 2 mm or more in diameter. The number of apertures 12b is not particularly restricted, and the aperture units are preferred to be formed in the entire collar unit so as to enable visual confirmation from various angles, for example, and may be 3 to 8 in number. Furthermore, the entire collar unit 11 may have a mesh structure.

The apertures 12b may be formed by injection molding, laser molding, heat molding, and other methods. As for the shape of the apertures 12b, various configurations such as a circular shape, an elliptical shape, a start shape, a heart shape, and a clover shape can be adopted.

In the first modification, the configuration is such that apertures 12b are formed in the collar unit 12, and the operating hand can be visually confirmed via the apertures, however, the configuration may be such that, instead of the apertures 12b, a plurality of high light transmissive means each having a total light transmittance of 63% or more is formed in the region of the apertures 12b, whereby the hand operating the inner tube, etc. can be visually confirmed via the high light transmissive means.

The collar unit 13 according to the second modification is entirely semi-transparent, and concavities 13c recessed toward the hole 13a (central side) are formed on its circumference. By providing concavities 13c, the user can dispose the fingers along the concavities 13c and can grip the collar unit 13. Thus, the outer tube 2 can be inserted with fingers placed in the concavities 13c, the outer tube can be inserted at the appropriate position and angle inside the vagina, and the absorber 4 can be disposed easily at the appropriate position inside the vagina. Furthermore, by providing concavities 13c, even if the user has long finger nails or is wearing artificial nails, the nails can be directed in a direction away from the vaginal opening, and the nails can be prevented from coming in contact with the body of the user, such as the vaginal opening.

Furthermore, three concavities 13c are formed, and the interval between any two concavities 13c is the same, and the three concavities 13c are disposed at an angle of 120 degrees with respect to the center of the hole 13a. By providing the collar unit 13 in this way, the collar unit 13 can be gripped with three fingers, and the stability at the time of inserting the outer tube 2 can be improved. Finally, by setting the intervals between adjacent concavities 13c to be the same, the collar unit 13 can be held by uniformly applying force via the concavities 13c, and the stability at the time of gripping can be improved. Three or more concavities can be formed.

The concavities 13c may be formed by injection molding, laser molding, heat molding, and other methods. As for the shape of the concavities 13c, various configurations such as a streamline, a concave, an isosceles triangle, and a teardrop can be adopted.

Furthermore, in the collar unit 14 according to the third modification, the hole 14a in which the outer tube is inserted, and the apertures 14b that act as the light transmissive means are unified into one part. In FIG. 6, the hole 14a is disposed at the approximate center of the collar unit 14, and four apertures 12b are formed radially outward from the center. Thus, by unifying the hole 14a and the apertures 14b, the apertures can be formed simultaneously, and therefore, the manufacturing efficiency can be improved. Furthermore, the collar unit 14 is in contact with the outer tube 2 at four contact points, and because these four contact points are fitted in to the outer tube 2 as support points, the fitting balance improves, and the fitting state can be made stable. The collar unit may be performed with the unified opening by injection molding, laser molding, heat molding, and other methods. The unified opening may also be cut out from a preform of the collar unit.

Thus, needless to say, the present invention includes various embodiments not specifically described herein. Therefore, the technical range of the present invention is to be defined only by the inventive specific matter according to the adequate claims from the above description.

For example, the collar unit may have an outer shape other than a circular shape, and may have a polygon shape such as a rectangular shape or a triangular shape, or a heart shape and a star shape, or an elliptical shape, or the shape of the head of animals in a plan view. For example, by shaping the outer shape of the collar unit into a heart shape and the shape of the head of animals, a medical device like a tampon can be beautified by the collar unit. Thus, the depressing feeling of the user associated with menstruation can be eased, and the willingness to use such a medical device the next time can be increased.

Furthermore, by adding a color to the collar unit, the decorative effect can be improved further. The color added to the collar unit is not particularly limited, for example, red and pink color can be added to a heart-shaped collar unit, and brown color can be added to a collar unit shaped like the head of a bear. Finally, by coloring the collar unit, the collar unit can be distinguished, and the handling becomes easy particularly in dimly lit places, such as toilets.

Furthermore, the outer circumferential shape of the collar unit is curved in some embodiments. In such a curved shape, sharp angular portions are not included, although a linear side may be included. By shaping the outer circumference of the collar unit as curved, the feeling of gripping the collar unit experienced by the user can be mellowed down.

### EXAMPLES

Hereinafter, the evaluation of visibility of the collar unit in accordance with some exemplary embodiments of the present invention is explained in detail with reference to examples and comparative examples. The present invention is not limited to the examples given below.

### Evaluation Contents

By using collar units having different values of the total light transmittance according to examples 1 through 4 and comparative examples 1 and 2, the visibility was evaluated concerning whether or not the hand, for example, at the dorsal side could be recognized via the collar unit.

(Method of Measuring the Total Light Transmittance)

The collar unit is held between test piece scissors, the test bench is moved such that the light-receiving hole is positioned in the center of the bunch of light rays, and the cover of the optical testing chamber is closed. Next, the total light transmittance is measured. The total light transmittance was measured at six locations for each collar unit, and the average value was calculated.

(Measurement device for the total light transmittance)

A color measuring differential colorimeter (Model NDH-300A manufactured by Nippon Denshoku Industries Co. Ltd.) was used.
Measurement method: Conforms to the measurement method A of JIS K7105
Measurement light source: 12-V 4-A halogen lamp with a lifespan of 2000 hours
Optical path system: Flicker photometry of the total light transmittance and diffuse transmittance
Light-receiving element: One silicon photocell conforming to JIS K 7105 was used
Filter: Glass filter Y
Measurement area: diameter 12 mm
Power supply: AC100V, 50/60 HZ

(Evaluation Method)

By using a collar unit according to examples 1 through 4 and comparative examples 1 and 2, it was determined whether or not the dorsal side of the collar unit could be visually confirmed. The number of monitored persons was five. In each example and comparative example, five monitored persons determined the visibility. If the monitored persons decided that the dorsal side could be visually confirmed, the dorsal side was determined to be recognizable, and if the monitored persons decided that the dorsal side could not be visually confirmed, the dorsal side was determined to be unrecognizable.

(Evaluation Results)

The evaluation results are shown in Table 1.

TABLE 1

| | Comparative | | Comparative | | | | | | | | | |
|---|---|---|---|---|---|---|---|---|---|---|---|---|
| | Example 1 | | Example 2 | | Example 1 | | Example 2 | | Example 3 | | Example 4 | |
| Transmittance | 1 | 57 | 1 | 61.09 | 1 | 63.52 | 1 | 69.11 | 1 | 72.5 | 1 | 85.63 |
| (%) | 2 | 56.99 | 2 | 61.09 | 2 | 63.44 | 2 | 69.11 | 2 | 72.48 | 2 | 85.57 |
| | 3 | 56.98 | 3 | 61.1 | 3 | 63.37 | 3 | 69.1 | 3 | 72.47 | 3 | 85.52 |
| | 4 | 56.98 | 4 | 61.1 | 4 | 63.31 | 4 | 69.09 | 4 | 72.45 | 4 | 85.49 |
| | 5 | 56.97 | 5 | 61.1 | 5 | 63.26 | 5 | 69.08 | 5 | 72.43 | 5 | 85.46 |
| | 6 | 56.98 | 6 | 61.1 | 6 | 63.21 | 6 | 69.06 | 6 | 72.5 | 6 | 85.42 |
| | Average | 56.98 | Average | 61.10 | Average | 63.35 | Average | 69.09 | Average | 72.47 | Average | 85.52 |

| | | | | | | |
|---|---|---|---|---|---|---|
| Recognized by | 0 | 0 | 2 | 3 | 5 | 5 |
| (Number of | | | | | | |
| Persons) | | | | | | |

Two or more monitored persons determined that the collar unit according to examples 1 through 4 was recognizable, and it was understood that the dorsal side could be visually confirmed via the collar unit. Therefore, the total light transmittance of the collar unit is desired to be 63% or more. Furthermore, all of the five monitored persons determined that the collar unit according to example 3 and example 4 was recognizable. Therefore, the total light transmittance of the collar unit is further desired to be 72% or more.

This application claims the benefit of Japanese Application No. 2011-033315 the entire disclosure of which is incorporated by reference herein.

1. An tampon applicator, comprising:
an outer tube which contains an absorber therein, of which the one side is provided with a push-out aperture by which the absorber is to be pushed out, and of which the other side is provided with a grip tube unit;
an inner tube which is inserted inside the grip tube unit, and which is movable towards into the outer tube to push the absorber to the outside through the push-out aperture; and
a collar unit extending radially outwards from an outer circumferential surface of the grip tube unit of the outer tube is included, and having a light transmissive means that enables the grip tube unit side to be visually confirmed from the push-out aperture side of the outer tube via the collar unit.

2. The tampon applicator according to claim 1, wherein a total light transmittance of the light transmissive means is 63% or more.

3. The tampon applicator according to claim 1, wherein
the collar unit is either semi-transparent or transparent, and
the light transmissive means is formed in the entire collar unit.

4. The tampon applicator according to claim 1, wherein the light transmissive means includes at least an aperture formed in the collar unit.

5. The tampon applicator according to claim 4, wherein
the collar unit includes a hole in which the outer tube is insertable is formed, and
the aperture and the hole are unified into one opening.

6. The tampon applicator according to claim 2, wherein the total light transmittance of the light transmissive mean is 72% or more.

7. The tampon applicator according to claim 2, wherein the total light transmittance of the light transmissive means is 90% or less.

8. The tampon applicator according to claim 1, wherein
the collar unit includes a hole in which the outer tube is inserted, and
the light transmissive means includes multiple apertures arranged around the hole.
